(19) United States
(12) Patent Application Publication
(10) Pub. No.: US 2022/0208315 A1
(43) Pub. Date: Jun. 30, 2022

(54) METHOD AND SYSTEM FOR OBTAINING, CONTROLLING, ACCESSING AND/OR DISPLAYING PERSONAL GENETIC IDENTIFICATION INFORMATION

(71) Applicants: Duangkamol STROHL, Danville, CA (US); Paul McLAREN, Senoia, GA (US)

(72) Inventors: Duangkamol STROHL, Danville, CA (US); Paul McLAREN, Senoia, GA (US)

(73) Assignee: STR-ID, Inc., Dover, DE (US)

(21) Appl. No.: 17/555,968

(22) Filed: Dec. 20, 2021

Related U.S. Application Data
(60) Provisional application No. 63/131,626, filed on Dec. 29, 2020.

(51) Int. Cl. G16H 10/40; G06F 16/27; C12Q 1/6876; G16B 50/40; G16B 50/30; G16B 50/20

(52) U.S. Cl. CPC G16H 10/40; G06F 16/27; G16B 50/20; G16B 50/40; G16B 50/30; C12Q 1/6876

(57) ABSTRACT

A method and system for obtaining and controlling genetic identification information are disclosed. The method includes providing personal information of a registrant to a secure website using an electronic communication device, taking a genetic material-containing sample from the registrant, providing the sample to a genetic material analysis facility, analyzing short tandem repeat (STR) regions of the genetic material at a plurality of loci to produce a genetic identity for the registrant, recording the personal information and the genetic identity in a blockchain ledger, and enabling the registrant to display on another electronic communication device a code corresponding to the genetic identity. The system includes a genetic material sampling kit, a short tandem repeat (STR) analysis kit, and electronic communication device(s) configured to enter the registrant's personal information, record the personal information and the genetic identity in the blockchain ledger, and display a code corresponding to the genetic identity.

### RELATED APPLICATION(S)

This application claims priority to U.S. Provisional Pat. Appl. No. 63/131,626, filed Dec. 29, 2020, incorporated herein by reference.

### FIELD OF THE INVENTION

The present invention generally relates to the field(s) of obtaining, controlling and accessing genetic identification information. More specifically, embodiments of the present invention pertain to novel methods and systems for obtaining, storing, controlling and accessing genetic identification information, particularly those using short tandem repeat analysis and blockchain data/transaction storage and retrieval, and methods of making and using the same.

### DISCUSSION OF THE BACKGROUND

Short tandem repeat (STR) analysis is an informative approach to genetic identification and is commonly associated with DNA testing in forensic laboratories, paternity disputes or missing persons cases. Among the 3 million or so DNA bases that do not code for proteins are regions with multiple copies of short repeating sequences of these bases, which make up the DNA backbone (for example, TATT). These sequences repeat a variable number of times in different individuals. Such regions are called “variable number short tandem repeats,” and they are the basis of STR analysis. A collection of these repeating sequences at different loci in the genome can give nearly irrefutable evidence statistically of a person's identity because the likelihood of two unrelated people having the same number of repeated sequences at these loci becomes increasingly small as more loci are analyzed.

Currently, consumers generally lack the ability to easily participate in managing their identity before, during and even after emergency events. To the best of the inventor's knowledge, “pre-event” preparation for human casualty using a pre-analyzed DNA identification system does not exist. For example, traditional forms of identification (ID), such as driver's licenses, passports, birth certificates, etc., can be manipulated or stolen, and are often simply outdated (e.g., for digital purposes). Thus, a need exists to be able to identify a person during an emergency or catastrophic event (e.g., an accident or crime scene, a natural disaster, during war/conflict, after an act of terrorism, etc.) conclusively, reliably, safely (e.g., using any necessary or desired cybersecurity and/or digital traceability), verifiably, and easily, while still protecting the person's privacy.

For example, according to one report, more than 800,000 children go missing each year in the United States (Goldberg, B., “Missing Children in U.S. Nearly Always Make It Home Alive,” Reuters News Service, Apr. 26, 2012). That corresponds to one child gone every 40 seconds. Major disasters, both natural and man-made, are no longer uncommon. Casualties and death are an unfortunate consequence of military, defense, law enforcement and other first response activities, and it is often undesired to ask next-of-kin to identify persons who recently died in service to their country or community.

The time period for identification of persons adversely affected by accidents or catastrophic events is often unacceptably long. Furthermore, historically, the process of identification using genetic information generally takes a great deal of time due to the need to find family members for appropriate sampling. If the family members do not live in the immediate area, multiple agencies generally are involved in locating family members and obtaining samples. If the family members live in another country, the time and number of agencies involved increases significantly. For example, it took the California Office of Emergency Services (OES) about 10 days to identity 34 victims following a fire on Sep. 2, 2019 on the Conception during a planned three-day dive trip, even though the names of all victims were known and recorded prior to the trip. The effort also involved multiple agencies (the Federal Bureau of Investigation [FBI], the Los Angeles County Medical Examiner, the Santa Barbara County Sheriff's Office, and the Sacramento County Coroner), and has the advantage of rare on-site mobile rapid DNA technology. It is not always possible to involve so many agencies or to have such valuable resources available in every major accident or mass casualty event.

Relatively lengthy identification procedures are also inconsistent with some religious customs and/or practices for the treatment of the recently deceased. Many religious groups around the world believe in the sacredness of the human body after death. Some religious practices do not approve of autopsies because they desecrate the sacred body. Identification via DNA (even when a rapid DNA test is used) is still hampered by the process of locating the appropriate family member for a reference sample. If family members cannot be located quickly or are no longer alive, the time increases for the identification, an autopsy may be deemed necessary, and neither the decedent's nor the family's religious beliefs are upheld.

Despite millions of dollars being poured into human identification efforts, families often must wait for days, months or even years to confirm the identity of their loved ones. If successful, movements to defund law enforcement could have an adverse impact on human identification efforts, making the process slower and more difficult for everyone involved.

Contracts, transactions, and associated records are fundamental constructs in our economic, legal, and political systems. They protect assets and set organizational boundaries. They establish and verify identities and chronicle events. They govern interactions among nations, organizations, communities, and individuals. They guide managerial and social action, and yet these critical tools (and the bureaucracies formed to manage them) appear to have had difficulty keeping up with modern economies' digital transformation.

Blockchain promises to solve at least some problems with managing contracts, transactions, and the information associated therewith. The technology at the heart of bitcoin and other virtual currencies, a blockchain is a data structure that enables creation of an open, distributed digital ledger that can record transactions between two parties efficiently and in a verifiable and permanent way. The ledger can be shared among a network of independent parties and also be programmed to trigger further transactions automatically.

This “Discussion of the Background” section is provided for background information only. The statements in this “Discussion of the Background” are not an admission that the subject matter disclosed in this “Discussion of the Background” section constitutes prior art to the present disclosure, and no part of this “Discussion of the Background” section may be used as an admission that any part of this application, including this “Discussion of the Background” section, constitutes prior art to the present disclosure.

### SUMMARY OF THE INVENTION

In one aspect, the present invention concerns a method of obtaining and controlling genetic identification information, comprising providing personal information of a registrant to a secure website using a first electronic communication device, taking a genetic material-containing sample from the registrant, providing the genetic material-containing sample to a genetic material analysis facility, analyzing short tandem repeat (STR) regions of the genetic material at a plurality of loci to produce a genetic identity for the registrant, recording the personal information and the genetic identity in a blockchain ledger, and enabling the registrant to display on a second electronic communication device a code corresponding to the genetic identity. The first and second electronic communication devices may be the same device or different devices. In various embodiments, the first and second electronic communication devices are independently selected from a smart phone, a personal computer, a tablet computer, and a work station.

The personal information may comprise at least two of a name, an address, a government-issued identification number, and a photograph of the registrant. For example, the government-issued identification number may comprise a social security number, a driver's license number, or a passport number.

In some embodiments, the method may further comprise (i) encrypting the personal information and the genetic identity of the registrant prior to recording the personal information and the genetic identity in the blockchain ledger and/or (ii) registering the registrant for a service comprising the STR region analysis, the personal information/genetic identity recordation, and genetic identity code display enablement. In a further embodiment, the method may further comprise ordering a home genetic material sampling kit on the website. The home genetic material sampling kit may comprise a vial or tube, written instructions for taking the sample, and/or a pre-addressed envelope or box for sending the genetic material-containing sample to the genetic material analysis facility. In an even further embodiment of the method, taking the genetic material-containing sample may comprise placing the genetic material-containing sample in the vial or tube, then placing the vial or tube containing the genetic material-containing sample in the pre-addressed envelope or box.

In various embodiments, taking the genetic material-containing sample from the registrant comprises collecting the registrant's saliva in the vial or tube, swabbing an inner surface of the registrant's mouth or nose, or pricking/puncturing the registrant's skin and collecting one or more drops of the registrant's blood on a swab or a piece of absorbent paper. In some such embodiments, the method further comprises (i) the registrant certifying or confirming that they collected the DNA sample or (ii) a third party (who may be registering or collecting samples from one or more other individuals, such as a minor, a disabled person, an employee or a government services beneficiary) certifying or confirming that they have the authority to collect the DNA sample(s) of the registrant(s). In other or further embodiments, providing the genetic material-containing sample to the genetic material analysis facility comprises shipping the genetic material-containing sample to the genetic material analysis facility in an envelope, sleeve, tube or box.

In some embodiments, analyzing the STR regions of the genetic material comprises extracting DNA from the genetic material, optionally quantifying the DNA, amplifying the DNA at multiple STR loci, separating and sizing amplified STR alleles, and interpreting a profile of the separated and sized STR alleles. The method may further comprise labelling the amplified STR alleles, during or after amplification. In some cases, the DNA is amplified at 20 or more STR loci, and separating and sizing the amplified STR alleles may comprise (i) separating the labelled, amplified STR alleles by gel electrophoresis or capillary electrophoresis, (ii) irradiating the labelled, amplified STR alleles with light that causes the labelled, amplified STR alleles to fluoresce or luminesce, and (iii) measuring a fluorescence or luminescence of the irradiated, labelled, amplified STR alleles.

In various embodiments, the method may further comprise allowing the registrant to access entries in the blockchain ledger containing the personal information and the genetic identity, authenticating an identity or personal information of the registrant using the registrant's genetic identity, and/or enabling the registrant to authorize third parties to access the code on a third electronic communication device. In the latter embodiment, the method may even further comprise accessing the code using one of the first, second and third electronic communication devices. The third electronic communication device may be identical to, the same as, or different from one or both of the first and second electronic communication devices. However, in general, the method may further comprise accessing the code using one of the first and second electronic communication devices.

Another aspect of the present invention related to a system for obtaining and controlling genetic identification information, comprising a genetic material sampling kit, a short tandem repeat (STR) analysis kit, a first electronic communication device configured to enter personal information of the registrant to a secure website, a second electronic communication device configured to record the personal information and the genetic identity in a blockchain ledger, and a third electronic communication device configured to display a code corresponding to the genetic identity. The genetic material sampling kit comprises a sealable container configured to sealably contain a sample containing genetic material of a registrant, written instructions for taking the sample from the registrant and placing the sample in the sealable container, and a pre-addressed envelope or box for sending the sample in the sealable container to a genetic material analysis facility. The STR analysis kit comprises a plurality of primers for copying STR regions in the genetic material at a plurality of loci, and a mixture containing a genetic material polymerase, buffers, and dNTPs necessary to amplify the STR regions, compare the amplified STR regions with similar genetic identification information and produce a genetic identity for the registrant. The second electronic communication device is different from the first and third electronic communication devices, and the first and third electronic communication devices may be the same electronic communication device or different electronic communication devices.

The first electronic communication device may comprise a personal computer or a smart phone, which may be configured (e.g., with an app) to enter at least two of a name, an address, a government-issued identification number, and a photograph of the registrant as the personal information. In some embodiments, the first electronic communication device may be further configured to enable the registrant to (i) register for a service comprising an STR analysis and recordation of the personal information and genetic identity, and/or (ii) access and/or display a code corresponding to the genetic identity. The second electronic communication device may comprise a personal computer, a workstation, or a server, for example, and may be configured to offer (a) a service comprising an STR analysis of the genetic material, recordation of the personal information and genetic identity, and/or creation of a code corresponding to the genetic identity of the registrant, and/or (b) the genetic material sampling kit to the registrant. The second and/or third electronic communication devices may be further configured to authenticate an identity or personal information of the registrant using the registrant's genetic identity, and the third electronic communication device may be further configured to access the code from the blockchain ledger.

In various embodiments, the sealable container comprises a sealable plastic bag or a vial or tube with a cap or lid configured to seal an opening in the vial or tube, the STR analysis kit further comprises (i) a gel electrophoresis cassette/tray and a gel or (ii) a capillary electrophoresis capillary, the primers include a fluorescent or luminescent label, and/or the system further comprises a genetic analyzer. The gel electrophoresis cassette/tray and gel or the capillary electrophoresis capillary are/is configured to separate the amplified STR regions by size. In the latter embodiments, the STR analysis kit may further comprise a plurality of allelic ladders for the loci, wherein each of the allelic ladders has a predetermined size, and the allelic ladders are configured to calibrate sizes of the amplified STR regions to an STR repeat number.

The present invention offers a novel digital genetic (e.g., DNA-based) identity management system and method, where users have control of their identification information, in many cases substantially anywhere and/or substantially at any time. In many embodiments, the invention is human-centered, connects easily and directly to consumer/users and their electronic communication devices (e.g., smartphones), and leverages advanced privacy protection, genetic identification technology, and tamperproof blockchain technology to generate and/or authenticate an individual's “forever identification.” For example, the present invention can use the same human identification technology (e.g., STR) that is used and accepted by the U.S. Federal Bureau of Investigation (FBI) and law enforcement agencies around the world.

The present invention uses non-coded regions of individuals' DNA to distinguish a person's identity with substantially irrefutable accuracy, while protecting the individual's genetic privacy. The present system and method also enable compliance with many after-life religious procedures and/or customs. The present system and method remove the need to find family members for positive genetic identification, thereby saving time, money and resources for government agencies, as well as the families themselves (in addition to reducing the stress on the families), to locate and confirm the identity of their loved ones. This is very important especially in times of mass disaster, when resources are stretched thin.

### BRIEF DESCRIPTION OF THE DRAWINGS

FIG. 1 is a flow chart of an exemplary method of obtaining and providing genetic identification information in accordance with one or more embodiments of the present invention.

FIG. 2 is a diagram showing color-separated panels for an allelic ladder from a commercially-available STR analysis kit used for STR test calibration.

FIG. 3 is a block diagram showing components of a generic system for obtaining and accessing/providing genetic identification information in accordance with one or more embodiments of the present invention.

FIG. 4 is a diagram of an exemplary blockchain in accordance with one or more embodiments of the present invention.

FIG. 5 is a flow chart illustrating an exemplary method of managing genetic identification information in accordance with one or more embodiments of the present invention

FIG. 6 is a flow chart illustrating an exemplary genetic information recording process and exemplary ledger transactions using a public permission blockchain and blockchain network in accordance with one or more embodiments of the present invention.

FIG. 7 is a block diagram illustrating an exemplary personal and genetic information privacy protection system in accordance with one or more embodiments of the present invention.

FIG. 8 is a block diagram illustrating an exemplary blockchain with a distributed ledger for recording personal and genetic identification information and transactions related thereto in accordance with one or more embodiments of the present invention.

FIG. 9 shows a smartphone with an exemplary personal and genetic identification data retrieval page displayed thereon in accordance with one or more embodiments of the present invention.

FIG. 10 is a block diagram showing components of an exemplary PC/computer system suitable for use in the present system and method.

### DETAILED DESCRIPTION

Reference will now be made in detail to various embodiments of the invention, examples of which are illustrated in the accompanying drawings. While the invention will be described in conjunction with the following embodiments, it will be understood that the descriptions are not intended to limit the invention to these embodiments. On the contrary, the invention is intended to cover alternatives, modifications and equivalents that may be included within the spirit and scope of the invention. Furthermore, in the following detailed description, numerous specific details are set forth in order to provide a thorough understanding of the present invention. However, it will be readily apparent to one skilled in the art that the present invention may be practiced without these specific details. In other instances, well-known methods, procedures and components have not been described in detail so as not to unnecessarily obscure aspects of the present invention. Furthermore, it should be understood that the possible permutations and combinations described herein are not meant to limit the invention. Specifically, variations that are not inconsistent may be mixed and matched as desired.

For the sake of convenience and simplicity, the terms “user,” “consumer,” and “registrant” may be used interchangeably herein, but are generally given their art-recognized meanings. In general, wherever one such term is used, it also encompasses the other terms. Similarly, for convenience and simplicity, the terms “party” and “entity,” and separately, the terms “individual” and “person,” and the terms “information” and “data,” are, in general, interchangeable and may be used interchangeably herein, but are generally given their art-recognized meanings, and wherever one such term is used, it also encompasses the other terms. In addition, for convenience and simplicity, the terms “part,” “portion,” and “section” may be used interchangeably but these terms are also generally given their art-recognized meanings. Also, unless indicated otherwise from the context of its use herein, the terms “known,” “fixed,” “given,” “certain” and “predetermined” generally refer to a value, quantity, parameter, constraint, condition, state, process, procedure, method, practice, or combination thereof that is, in theory, variable, but is typically set in advance and not varied thereafter when in use.

One ultimate goal of the present invention is to facilitate preparedness in and/or among the general public regarding emergency identification, and at the same time, directly support law enforcement efforts, making the identification process faster, easier, and less expensive for society as a whole. Other goals include enabling fast and facile identification of individuals and/or ownership and traceability of digital assets (such as personal information in electronic form) in a safe and secure manner. The present invention aims to decrease pressure, stress and/or reliance on limited public and private resources, especially during a mass disaster event (whether natural or man-made), and perhaps most importantly, bring closure to grieving families more efficiently and/or less invasively. The present invention offers a novel solution for many religious groups for identifying the recently deceased without an autopsy, as such groups may value the freedom to honor their deceased based on their religious beliefs and practices.

In some aspects of the present invention, users can save another's life with the timely identification process of a decedent or person on life support. Many individuals and their families believe in and offer organ donation after death. If an individual is on life support or has very recently died, but they are not properly identified, the organ donation process is hampered. Many organ procurement agencies require the donating individual's clear and/or written consent (e.g., through a donation list) or the family's permission before death for organ donation. If the identity of the person is unknown or the agencies cannot locate the family, this lengthens the time before a donation can be completed. Organ donation is a time-sensitive process, and viability of organs decreases significantly with time. The present invention provides an advanced method of identifying an individual, saving time and possibly saving others' lives in when the individual has authorized organ donation. In some aspects of the present invention, the registrant may use the digital genetic (e.g., DNA-based) identity management system and method, for verifying identity in order to access specific electronic systems, such as but not limited to corporate computing, intranets, bank accounts, or on-line database storage.

Identity is an important part of society, but it is even more critical during exigent circumstances. In some aspects of the present invention, users and/or consumers own and manage access to and authorization of release of their identification information when they need it. The present system and method can completely avoid surveillance and invasions of privacy.

The present system and method, which may be considered as a kind of “DNA digitalization,” has global interconnected security applications because law enforcement agencies around the world use the exact same scientific bases for genetic identification. An individual's ID in the present system and method is a kind of “forever identification,” valid and relevant from the individual's birth to after the end of the individual's life. By recognizing that public security is, to some extent, the responsibility of the public, the present system and method can make the world safer for all, especially for the most vulnerable persons.

Exemplary Methods

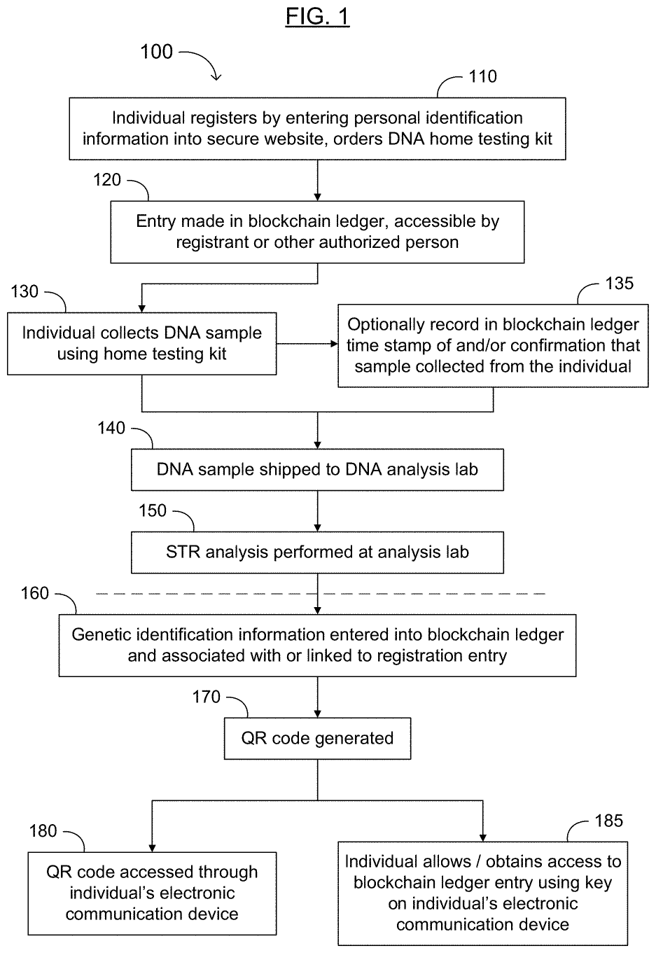

The present method may be divided into two processes: a testing process, and a digitalization/identification management process. An exemplary process for the present method is shown in the flow chart 100 in FIG. 1.

In a first step, an individual or user may register with an administrator of the blockchain registry at 110 by entering personal identification information on a secure website. The individual or user may be a person registering for themselves, for a minor or other person for whom they have custodial rights or power of attorney, or for another person who has granted authority to the individual or user. In the case of a child or another immediate family member without capacity (e.g., to form a contract), the individual or user may register for and act on behalf of such “family registered” users. A user may further register others (e.g., members of the same organization, such as a branch of the military, a police or fire department, a government agency, a relief organization, a church or religious organization, a hospital, a corporation, etc.) who give the user authority to register on their behalf.

The personal identification information entered into the secure website may include the registrant's name (legal name, and if different, birth name), birth information (e.g., date of birth and/or place of birth), biometric information (e.g., height, weight, color of eyes and/or hair), home address, mailing address, citizenship, driver's license information (e.g., driver's license number), passport or national identification information (e.g., passport number, country of issue, expiration date, etc.), marriage status, “multiple birth” status (i.e., the registrant is one of a set of twins, triplets, or other multiple birth group), or a combination thereof. Optionally, the individual may enter the registrant's race, religious affiliation, political affiliation, employment status, employer, health information (e.g., known chronic conditions, such as a disability, a chronic disease such as diabetes, hypertension, etc., presence of an internal health aid apparatus such as a pacemaker, known adverse drug reactions, etc.), next of kin, emergency contact information, educational status and information (e.g., high school[s] and university/ies attended, attendance dates, degree[s] obtained, etc.), etc. In addition, the individual may upload a photo of or other facial recognition information for the registrant, one or more fingerprints of the registrant, etc.

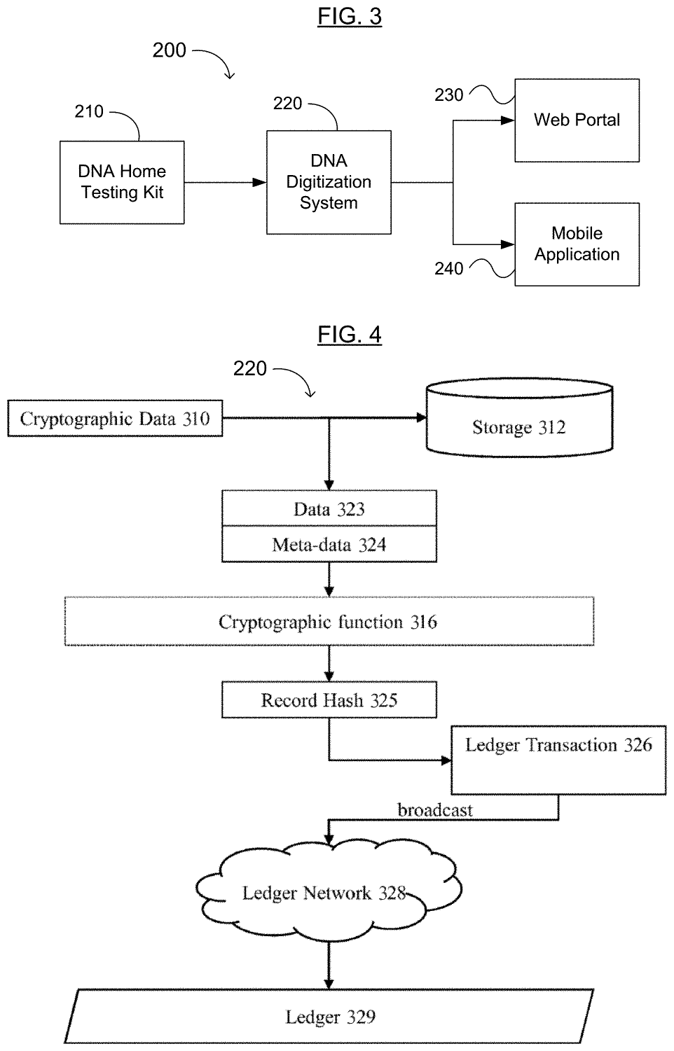

The individual may complete registration by ordering a DNA home testing kit at 110. The database/registry administrator generally confirms that no entry exists (e.g., in a blockchain register, other genetic identity database, etc.) for the registrant check prior to entry into the blockchain ledger at 120, and if necessary or desired, resolves any potential duplication of PII or genetic identification information to prevent any one individual from having more than one genetic identity. The database/registry administrator may also charge a fee for the kit and for shipping, and may collect any applicable tax. An entry is then made in a blockchain ledger at 120. The entry into the blockchain ledger is explained in greater detail with regard to FIGS. 3-4. The ledger entry is accessible to the registrant and to those authorized to access the ledger (e.g., on behalf of the registrant).

Alternatively, the registrant can simply enter identification information without ordering the DNA home testing kit, if the registrant's non-DNA identification information is to be maintained in the database/registry, but the benefits of access to the encrypted genetic identification information are lost. However, there may still be some benefits to storing non-DNA-based identification information in a blockchain registry.

The DNA home testing kit is then shipped to the registrant or the registrant's custodian or authorized agent (e.g., caretaker). When the kit is received by the registrant (or the registrant's custodian or authorized agent), a DNA sample is collected at 130 from the registrant in accordance with the instructions in the kit, and optionally, an entry is recorded in the blockchain ledger with a time stamp for the sample collection and/or a confirmation that the sample was collected from the individual whose information was entered into the secure website at 110. For example, the confirmation may comprise a certification or other written statement from the registrant confirming that they collected the DNA sample, or alternatively, from a third party confirming that they have the authority to collect the DNA sample(s) of the registrant(s). The third party may be registering (or collecting a sample from) one or more other individuals, such as a minor, a disabled person, an employee of the third party's employer, or a beneficiary of certain government services. In an alternative embodiment, limited PII (e.g., name, email address, date of birth, and optionally other basic identification information sufficient to create a unique identifier for the registrant) is collected at 110, and the remainder of the PII is collected at 130.

The sample is then shipped to the analysis lab at 140 for analysis. Typically, the kit will include a pre-addressed, postage-paid envelope or container for shipping the sample to the lab. In a further option, an entry is recorded in the blockchain ledger with a time or date stamp for shipment of the sample to the analysis lab. For example, the entry can be made by the registrant or third party (i.e., the user registering or entering information on behalf of another person) can use a camera on the registrant or user's communication device to generate one or more documentation photographs, such as of the bar code on the kit or the sample holder, the individual holding the sample ready to ship, etc. Such photographic entries may be useful to validate the identity of the registrant or user, at least in part.

At 150, the analysis lab conducts STR testing. Kits and equipment for performing STR testing are widely available. For example, STR test kits are available from Thermo Fisher Scientific Corporation under the Applied Biosystems brand (Waltham, Mass.), Promega Corporation (Madison, Wis.), Qiagen (Germantown, Md.) and others. Equipment such as genetic analyzers are available from Thermo Fisher Scientific and others.

Such commercially available kits, which typically provide premixed primers and a standard master mixture containing the polymerase, enzyme buffers, and dNTPs necessary to amplify STRs, simplify generation of STR profiles and provide results on a uniform set of core STR loci to make it possible to share genetic identification information and compare it with similar genetic identification information obtained from different samples. In fact, commercial kits are preferred in most analytical laboratories over in-house assays, even though the kits are more expensive. Commercially-available kits help simplify and standardize procedures, and remove the burden of PCR component quality control from the analysis lab. In addition, STR kits supply allelic ladders containing common STR alleles that have been previously characterized for the number of repeat units via DNA sequencing. These allelic ladders are used to calibrate PCR product sizes to STR repeat number for genotyping purposes. Genotype determination in subsequently processed samples is performed by comparing allele size (relative to an internal size standard) to a commercially provided STR kit allelic ladder with calibrated repeat numbers, which is sized according to the same internal size standard.

The process for STR testing includes sample collection, DNA extraction, DNA quantitation, PCR amplification of multiple STR loci, STR allele separation and sizing, STR typing and profile interpretation, and a report of the statistical significance of a match (if observed). Following PCR amplification, the overall length of the STR amplicon is measured to determine the number of repeats present in each allele found in the DNA profile. This length measurement is made via a sized-based separation using gel electrophoresis or capillary electrophoresis (CE). Each STR amplicon may be fluorescently labeled during PCR when either the forward or reverse locus-specific primer contains a fluorescent dye. By recording the dye color and migration time of each DNA fragment relative to an internal size standard, the size for each STR allele may be determined following its separation from other STR alleles. Commonly-used instruments for STR allele separation and sizing include the ABI PRISM 3100 and ABI PRISM 3500 genetic analyzers (available from Thermo Fisher Scientific Corporation under the Applied Biosystems brand).

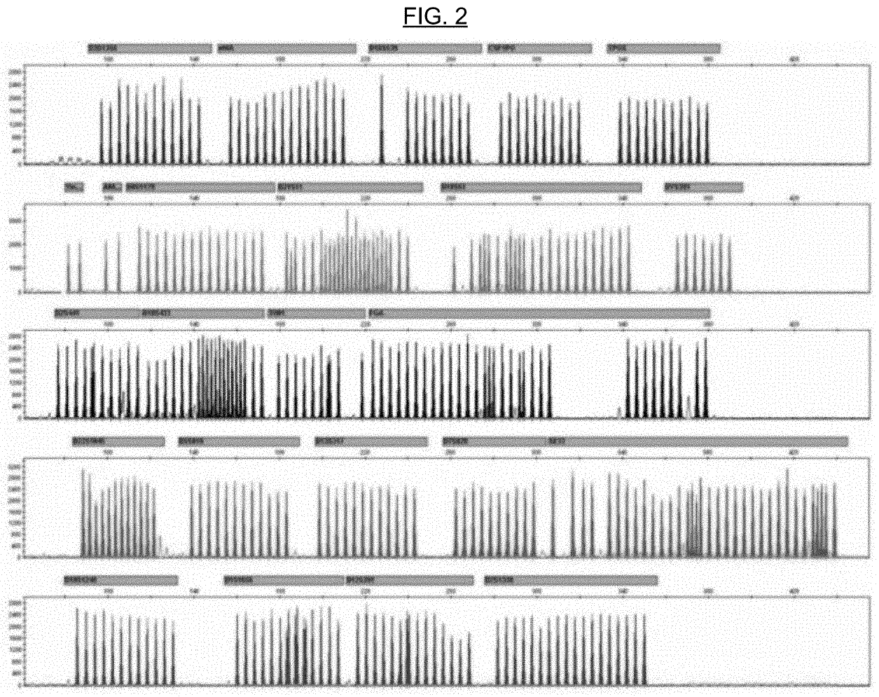

The result of the STR test is series or plurality of graphs or plots of the size of the repeated DNA segments at a number of predetermined loci, as determined by gel electrophoresis or capillary electrophoresis. Usually, the number of loci is from 11 to 25 (the greater the number of loci, the greater the confidence in the results; currently, the FBI requires at least 20 loci, and at least one commercial process [GlobalFiler, from Thermo Fisher] includes 24 loci), and the number of graphs or plots is based on the number of electrophoresis separations run in the test/analysis. For example, FIG. 2 shows five (5) color-separated panels for an allelic ladder from the AmpF/STR Globalfiler kit (available from Thermo Fisher) used for DNA size-to-short tandem repeat (STR) calibration. Genotype determination in a processed sample is performed by comparing allele sizes (relative to an internal size standard) in the processed sample to a STR allelic ladder (e.g., FIG. 2) with calibrated repeat numbers, sized according to the same internal size standard as the processed sample.

Referring back to FIG. 1, a digitalized format of the registered user's DNA analysis result (i.e., the genetic identification information) is reported directly to the registered user (e.g., the consumer), and entered into the blockchain ledger at 160. At the same time, the genetic identification information and/or the blockchain ledger entry are associated with the registration entry in the blockchain ledger. In some embodiments, the analysis lab encrypts the DNA analysis result before uploading (e.g., to the database/registry administrator or directly to the blockchain ledger) so that the genetic identification information is encrypted before it is entered into the blockchain ledger. In one variation of these embodiments, only the registrant (or, if authorized, the user) can decrypt the genetic identification information. This variation is somewhat critical to enabling self-sovereign identification (SSI) in the present methodology, and protects the data from access by third parties and unauthorized entities, who may do so innocently (e.g., using a law enforcement warrant) or with malicious intent. Once the registrant or authorized user receives the DNA analysis result, they can decrypt it using a decryption key (which may be a public key generated by the registrant, or the encryption key [or a complement thereof] programmed into a secure application provided by the database/registry administrator). In some examples, the DNA analysis result may be a further encrypted using a combination of the registrant's PII and/or DNA sequence before it is entered into the blockchain ledger. In a further embodiment, the registrant's identity may be validated using familial genetic information (e.g., comparing the registrant's genetic identification information to that of one or more members of the registrant's family).

At 170, the digital DNA analysis result (which may be previously encrypted) is embedded as a unique machine-readable icon or other symbol, such as a QR Code, a bar code, etc. For example, the machine-readable symbol may be a digital representation of the digital DNA analysis result and may comprise locus, allele and STR copy number information, converted to a digital format. In various embodiments, the digital format may comprise p characters, where p is an integer of (2^{q}+2^{r}), q is an integer or 5 or more, and r is 0 or an integer of one or more. In one example, p is 196. In a further embodiment, the digital format may be condensed or compressed using a conventional algorithm (e.g., to a smaller number of characters). The options for obtaining and testing the sample and for recording, reporting, displaying and otherwise using the test results are virtually unlimited.

For example, the registered user can share his/her genetic identification information with pre-approved individuals or entities, or present such information to the governmental authority in emergency situation (for example, by providing a decryption key to such individuals, entities or authorities). For example, genetic identification information can be presented to the governmental authority by showing the QR code or similar information displayed on the electronic communication device of the registrant or pre-approved individual or entity at 180. When the genetic identification information in the blockchain ledger is encrypted, the QR code or similar information is presented after decryption. Alternatively or additionally, genetic identification information can be shared with pre-approved individuals or entities (e.g., the registrant's emergency contact[s] or medical power of attorney holder) by sharing the access information and decryption key with the pre-approved individuals or entities at 185. Typically, the governmental authority (e.g., the police, FBI, coroner's office, etc.) will not receive authorization to use the registrant's genetic identification information for purposes unrelated to identification (e.g., in a criminal investigation).

In a further example, an optional location service (which may be available at no cost to the individual user or consumer) may enable the registrant (e.g., the user or consumer) to decide whether they want their location to be tracked. This can be done conventionally by an app on a smart phone or similar electronic communication device with geolocation-tracking hardware and software. Many currently-available apps track the device's geolocation, with or without the user's permission. In combination with required and/or appropriate authorizations and/or permissions (e.g., from the registrant, optionally recorded in a blockchain entry) to share the registrant's location or allow location tracking, location tracking enables law enforcement and/or others to determine a “last known location” for the registrant (which can, in some case, be a child) in case the registrant goes missing.

The genetic identification information may be managed in different ways. In one example, a private permission identification information management system is implementable as blockchain network, and can accept and retain personal identification information as well as genetic identification information. Personal identification information (which can be entered by the registrant or authorized user at the time of registration at 110 or 130 in FIG. 1) can include full given name, date of birth, place of birth, a “do not resuscitate” instruction, organ donor information, burial or cremation instructions, etc. Thus, the registrant's genetic identification in the present invention can be used to authenticate the registrant and/or the registrant's personal identification information. Other documents, such as copies of the person's birth certificate, marriage certificate, will, etc., may also be stored (e.g., as one or more additional blockchain entries). After the person's death, the person's death certificate may be associated with the registration in the blockchain ledger. The stored and/or associated documents may also be authenticated using the registrant's genetic identification.

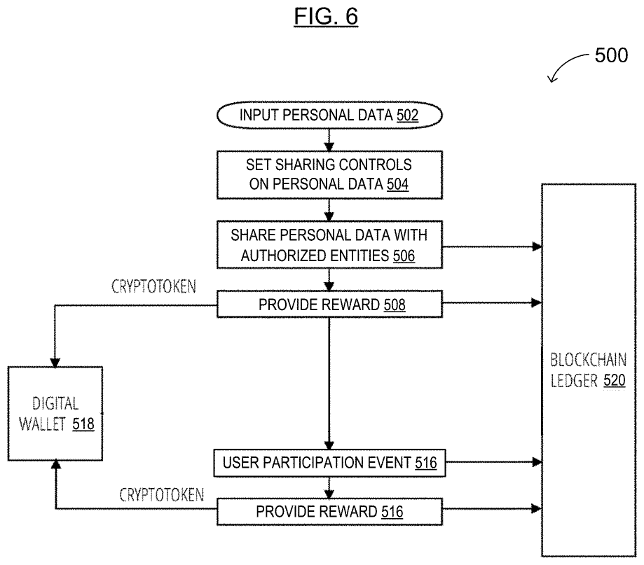

In another example, a public permission identification information management system (which is also implementable as blockchain network) can include a civic registry. Participation in the public permission identification information management system is voluntary, but may be encouraged through offers of dividends or other benefits (see, e.g., FIG. 6 and the discussion thereof herein). The civic registry may include the individual's political affiliation (e.g., voter identification), religious affiliation, one or more results of polling or asking the individual's opinions (e.g., voter integrity assurance, opinion polling result[s]), etc., to facilitate statistical analyses or other further data analysis.

An important advantage of the present invention is that an individual who has been forensically identified (e.g., via STR-ID analysis) can easily confirm the legality of their right to vote. Furthermore, the authentication aspects of the present invention ensure that any opinion survey and polling conducted using the genetic identification from the blockchain register to identify the survey participant is unbiased and is 100% certain that the participant is who they say they are and optionally is in the location that they claim to be in. In recent years, the results of certain political polling have resulted in questions about its accuracy and allegations of deceptiveness.

With blockchain-based transactions, contracts can be embedded in digital code and stored in transparent, shared databases, where they are protected from deletion, tampering, and revision. As a result, substantially every agreement, process, task and payment can have a digital record and signature that can be identified, validated, stored and shared. One aspect of the present invention keys the validation of the digital code to a unique genetic DNA record that may not be altered or superseded. Intermediaries such as lawyers, brokers and bankers may not be necessary for such activities. Individuals, organizations, machines, and algorithms can therefore freely interact with one another and conduct transactions with little friction. These are among the many advantages of blockchain.

FIG. 3 shows a block diagram of a system and/or hardware 200 for implementing a method of obtaining and providing genetic identification information in accordance with embodiments of the present invention. The system and/or hardware 200 comprises a DNA testing kit 210, a genetic information digitization system 220, a web portal 230 and a mobile application 240.

The DNA testing kit 210 may comprise a home testing kit as described herein. Alternatively, the test kit may be a sample-taking kit that is used commercially (e.g., in a medical office or other health care service provider facility, a testing service provider facility, a forensic lab, etc.).

The genetic information digitization system 220 is largely conventional. An example of a personal genetic identification management is shown in FIG. 4, which shows a blockchain distributed ledger 329 that stores encoded data relating to the registrant's genetic identification. The ledger 329 may be an immutable distributed ledger, and the blockchain may include, for example, a public blockchain and/or a private blockchain. In some embodiments, the storage 312 may be the same as the ledger 329. The system 220 in FIG. 3 provides safety and integrity for multiple records and events within the system 220, all within the parameters of a single ledger transaction 326 on the ledger 329.

Each new record (or combination of records) of a transaction in the storage medium 312 generates a ledger transaction 326 into the ledger 329, which allows anyone to verify and validate the existence and accuracy of the data entry. One embodiment of verification includes analyzing the cryptographic data 310 in combination with a digital signature for the ledger transaction 326 that is provided to the ledger 329. Advantageously, anyone can validate the existence of the information in the ledger transaction 326 based on the cryptographic data 310 using the storage 312 and the ledger 329. As shown in FIG. 3, the identification data 310 is stored in the storage 312, while also being divided into core data 323 and metadata 324. Metadata 324 is generally (but not always) not present within the cryptographic data 310, so core data 323 may be equal to the cryptographic data 310. Metadata 324 can be derived from external sources (not shown) and/or determined from other variables (e.g., timestamps). Both the core data 323 and the metadata 324 can be processed using the cryptographic function 316.

A record hash 325 is generated from the metadata 324 and the core data 323. The record hash 325 is distributed to the ledger transaction 326 as additional information. For a blockchain transaction, the record hash 325 is written into an ‘OP RETURN’ field of the ledger transaction 326. The ledger transaction 326 is broadcast over a ledger network 328. As soon as a new block (reflecting the transaction) is created on the ledger 329, the record(s) which the system 220 has placed within the ledger transaction 326 is/are secured inside the ledger 329 itself. In other words, when the ledger transaction 326 is in the block, it is difficult or impossible to change it or tamper with it, so it is difficult or impossible to change its history. Anyone in possession of the corresponding raw data can produce the cryptographic data 310, check its existence within the storage 312, and validate/verify information using the ledger 329.

Furthermore, in some embodiments, the storage medium 312 does not maintain data in its original or open form. In contrast, the raw data can be first processed through the cryptographic function 316 as shown in FIG. 3. This is advantageous in that hashed stored data cannot be reverse-engineered back to its original form, even if a hacker were to obtain access to the hashed data. In some embodiments, the personal genetic identification management system 220 can have at least one processor (e.g., in the registrant's electronic communication device) configured to perform cryptography primitives on identification information/data sets (e.g., the raw data and/or the cryptographic data 310).

Any input into the storage 312 as described herein may be followed by the generation of one or more ledger transactions 326 made in the ledger 329 as shown in FIG. 4, to provide a fully secured and trusted way of immutable data storage, validation and/or verification, and authentication. As used herein, the term “immutable data” refers to data that, once originated, never changes (e.g., names of birth parents, date of birth, biological sex, birth name, multiple birth status, place of birth) or that that is difficult to manipulate, for example, even for a system administrator, after the data has been written to a blockchain.

Each individual user of the system 220, such as a registered user or an authorized user, can be issued with a cryptographic secret key (such as a private key), which in some embodiments is relatively long. In some embodiments, the cryptographic secret key can comprise a Rivest-Shamir-Adleman (RSA) key, an elliptic curve cryptography (ECC) key, or the like. The known features of ECC enable this type of key to be split into a plurality of independent parts (factors). These factors can be of any nature, such as tokens, passwords, biometric data, pin-codes and the like, but are not limited to these examples.

In one common model for how blockchain works, there are five basic principles underlying blockchain technology. First, a distributed database is used. Each party on a blockchain has access to the entire database and its complete history. No single party controls the data or the information therein. Every party can verify the records of its transaction partners directly, without an intermediary.

Second, transactions and communications are conducted by peer-to-peer transmission. In other words, communication occurs directly between peers, instead of through a central node. Each node in a transaction or communication stores and forwards information to all other nodes in the transaction or communication.

Third, transactions on a blockchain are transparent, but the participants are not easily identified (i.e., there is pseudonymity). Every blockchain transaction and its associated value are visible to anyone with access to the system containing the blockchain. Each node, or user, on a blockchain has a unique 30-plus-character alphanumeric address that identifies the node/user. Users can choose to remain anonymous or provide proof of their identity to others, at the user's option. Transactions occur between blockchain addresses.

Fourth, records on a blockchain are irreversible. After a transaction is entered in the database and the accounts are updated, the records cannot be altered, because they are linked to every transaction record that came before them (hence the term “chain”). Various computational algorithms and approaches are deployed to ensure that the recording on the database is permanent, chronologically ordered, and available to all others on the network.

Fifth, blockchains use the computational logic available in the network/system that includes them. The digital nature of the blockchain ledger means that transactions recorded therein can be tied to computational logic, and in essence, programmed. As a result, users can set up algorithms and rules that automatically trigger transactions between nodes.

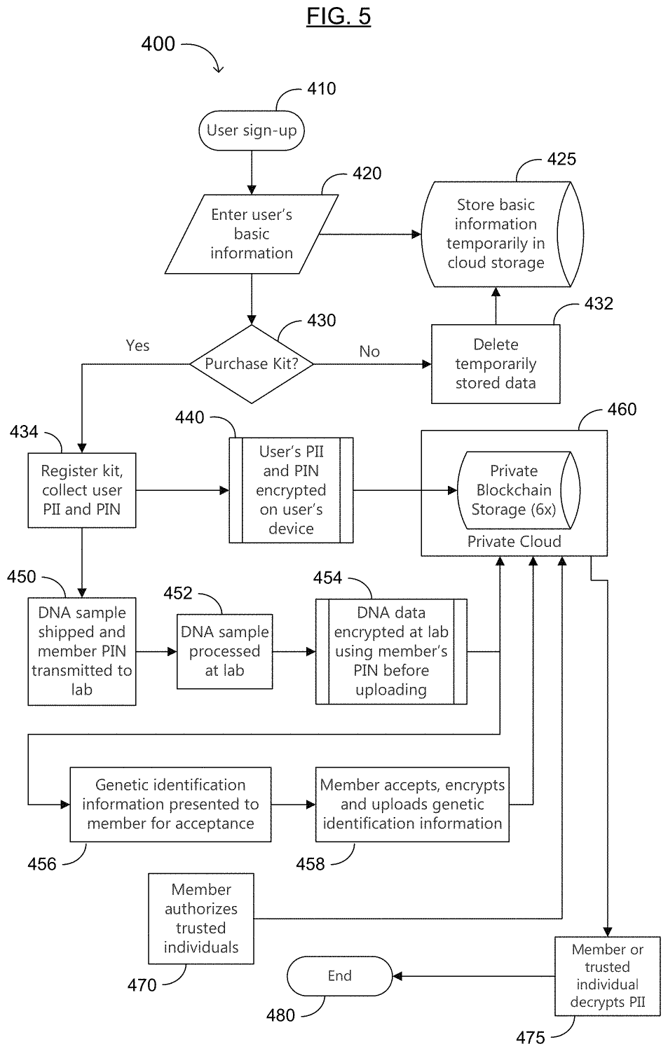

FIG. 5 shows a flow chart 400 for another exemplary method in accordance with embodiments of the present invention. The flow chart 400 is consistent with the flow chart 100 in FIG. 1, but may contain some variations and/or details that are not present in or discussed with regard to FIG. 1.

The method of FIG. 5 starts at 410 when the user signs up, for example, on a website or using an app. Typically, signing up comprises entering an email address or other personal communication information (e.g., a mobile phone number, a social media handle or username, etc.) of the user, and optionally, requesting information about services related to the method.

At 420, the basic personal information of the user is entered (e.g., into fields on a secure page of the website or into fields in the app). The personal information entered is generally a subset of the personal identification information (PII) for the flow chart 100 of FIG. 1, and may include the registrant's name, birth information, home address, mailing address, citizenship, etc., and optionally the user's employer or other organizational affiliation. The user's basic information is then stored temporarily in a cloud data storage system at 422.

At 430, the user decides whether to purchase a kit (e.g., a home testing or sample collection kit) as described herein. If the user decides not to purchase the kit, the data temporarily stored in cloud storage is deleted at 432. However, when the user purchases the kit, the kit (e.g., its serial number or other unique identifier) is recorded on a blockchain register at 434, and the user's PII and personal identification number (PIN) are collected (e.g., by entry into fields on the website or app). The user's PII may be selected from the personal identification information for the flow chart 100 of FIG. 1, other than the basic information entered at 420. The user also selects and enters a PIN, which may be n characters in length, where n is an integer of 6 or more (e.g., 6, 8, etc., optionally up to 12, 16, 20 or 24). The characters may be numbers, letters, or a combination thereof, optionally with one or more special characters, such as @, #, $, \hat{ }, &, *, a punctuation mark, etc. Also, after purchasing the kit, the user may be reclassified as a “member” or registrant.

At 440, the member's PII and PIN are encrypted on the device into which the PII and PIN were entered. Data entering the present genetic identification management system (including that entered at the user device) is encrypted using a local application on the local device (in this case, the user device) before uploading into the system. The algorithm(s) used to encrypt the PII and PIN may comprise one or more conventional (e.g., industry-standard, current best practice, etc.) algorithms, such as Asymmetric Encryption (AE), Advanced Encryption Standard (AES) or Blowfish, or a public key cryptography algorithm such as RSA. The encryption key is generated using a symmetric encryption technique, where the originator (e.g., the user or member) creates a key and shares it with an entity (e.g., the DNA testing facility, the individual whose genetic identification is being entered and managed, etc.) that needs to upload data on their behalf. For example, when a member submits their DNA sample to a lab or other testing facility along with their key at 450, the lab/testing facility uses the key to encrypt and upload the data at 454. In another example, when a government agency or other organization (e.g., a corporation) sets up an account for an individual in the organization using a key, the login information is sent to the individual (member) with the key as access. The individual (member) then uses their original key (e.g., PIN) to accept the data into an account associated with the individual (member) at the genetic identification management service provider at 456 (i.e., after uploading at 454, but prior to storage in the private blockchain ledger at 460). After acceptance at 458 (described in more detail below), the encrypted data is stored in the private blockchain register at 460. In one embodiment, the member is allowed to use the PIN once and only once (e.g., given a one-time use of the PIN) to decrypt, and thus accept, the DNA testing data. On uploading to account and/or upon storage in the private blockchain data storage system at 460, the information undergoes further encryption utilizing one or more standard blockchain hashtag algorithms.

In greater detail, at 450, the user/member takes a DNA sample using the kit as described herein and ships the sample along with their PIN to the DNA testing facility (e.g., laboratory). Alternatively, the kit (sample) may be physically shipped to the DNA testing facility, and the PIN may be transmitted electronically (and optionally, securely) to the DNA testing facility. At 452, the DNA sample is processed at the lab as described herein (e.g., STR tested) to obtain a digitalized version of the registered user's genetic identification information (i.e., a code based on the graphs or plots of the user's repeated [STR] DNA segments at a number of predetermined loci). This digitalized DNA data is encrypted at the lab (e.g., using one or more AE, AES and/or RSA encryption algorithms) using the user/member's PIN as an encryption key at 454 before it is uploaded to the private blockchain register in a private cloud storage system at 460. For even greater security, on data acceptance (e.g., in the private blockchain register at 460), all data may be decrypted, then re-encrypted using one or more AE or RSA algorithms.

At or about the same time that the encrypted genetic identification information is uploaded to the private blockchain register, it is also presented to (e.g., transmitted to, made available through the secure website or app, etc.) the member at 456 for acceptance. The member may accept the genetic identification information at 458 by decrypting it (e.g., using the PIN as a decryption key), viewing and confirming the acceptability of the decrypted genetic identification information (e.g., by checking a box on the secure website or app, sending a message to the genetic identity information service provider or administrator, etc.), re-encrypting the decrypted genetic identification information (e.g., using one or more standard and optionally embedded encryption algorithms on the secure website or app), and uploading the encrypted genetic identification information to the private blockchain register at 460.

At 470, the member may share the encrypted PII data stored at 460 with trusted individuals, groups or entities, with public keys being generated on the member's device as needed (e.g., to be provided to the trusted individual, group or entity, for either a single use or for multiple or recurring uses). In some embodiments, the user/member authorizes a particular device to receive the key (e.g., based on a random sequence of the user's/member's PII/DNA). The trusted individual, group or entity then uses the authorized device to enter a secure website (which may be the same as or different from that used by the user to register the member) or application programmed to enable access to (e.g., decrypt) the encrypted data by the trusted individual, group or entity.

At 470, the assignment and distribution of public keys (as selected by the member) to the trusted individuals, groups or entities is managed, for example using the secure website (which may be the same as or different from that used by the user to register the member) or application. The information by which a member or user accesses the system (typically a combination of email, password and system-determined PII multi-factor authentication data from the registrant's PII entries) is used to validate entry into the secure website or application and authorize generation of public keys. In one embodiment, the decryption key is generated from a unique combination of the user's/member's PII and a fragment or sequence of the member's DNA (e.g., an n-character sequence generated using a random sequence of the member's DNA) as a seed. One benefit of the DNA-as-a-seed approach is that the member's DNA cannot be changed, unlike a driver's license number or passport number. This decryption key may be similar to an authorization code transmitted by the owner or administrator of a secure website to an authorized individual for access to the secure website. The random sequence may be 6 or more bases long (e.g., 8 or more, 10 or more, 12 or more, etc.), up to about 100 bases long. Although there is no technical upper limit to the number of bases in the random sequence, typically no more than 50-60 bases are necessary or desired in the sequence.

The member or the trusted individual, group or entity then decrypts and accesses the encrypted PII (e.g., using the secure website or application and the public key) at 475. The decryption of PII and DNA may occur at later date, as long as trust is not revoked by the registrant, and may be utilized subsequent to death of the registrant to assist in establishing the identity of the deceased. In general, the key for decryption resides with the authority controlling the genetic identification information (e.g., the individual member when the individual member registers themselves, a corporation or governmental agency, bureau or other entity when the corporation or government entity registers the member, etc.). The method 400 may then end at 480, or return to 470 when the member wishes to generate and/or distribute another public key and authorize another trusted individual, group or entity.

Returning to 460, the member's encrypted PII data may be segmented in the private blockchain storage system so that only specific subsets of the member's data (e.g., member name, photograph, Social Security number or copy of the member's Social Security card, a completed W2 form, a signed non-disclosure and/or employment agreement, emergency contact information, shareable and/or confidential documents such as intellectual property documents or trade secret information [e.g., a customer list, sales strategy, etc.], security information such as permissions to access certain systems, etc.) are available to designated ones of the trusted individuals, groups or entities. More sensitive data, such as a member's genetic identification information, may be subject to additional protections that include converting the industry standard DNA information exchange format to a proprietary format which represents the member's raw or native DNA (e.g., in a format not generally recognizable by others, such as an n-digit-long numeric string that encodes the DNA information by digit and/or position in the string), optionally compressing the converted information, and then applying one or more industry-standard encryption algorithms to the compressed or uncompressed converted information. In some embodiments, the multi-level protection possible in the method 400 allows for generation of an individualized, unique identification number that may be used in genetic identification information management applications, the QR code for the member, or other identification methodology that does not expose the member's actual DNA information.

Further embodiments of the present invention relate to a unique architecture for managing self-sovereign identity (SSI) information (e.g., when the user/registrant is an individual) and PII (e.g., when the user/registrant is an organization). As described above with regard to the method/flow 400 in FIG. 5, PII data and transactional data (e.g., relating to registration) is encrypted and stored in the blocks of a private blockchain data repository. In this architecture, PII may (and typically does) include DNA information. Endpoint encryption (e.g., on the user's electronic communications device) may be based on a system-generated encryption key, generated from a unique combination of the user's/member's PII and a fragment or sequence of the member's DNA (e.g., an n-character sequence generated as described herein), using a random sequence of the member's DNA as a seed. This system-generated encryption key may function as a private key for the member.

As described above, the member generates a private/public security key when creating a PII profile. The private/public security key is used for encryption and decryption of the data in the system after the initial data upload. In some embodiments, only the member has access to the private key, and may decrypt data stored in the blockchain ledger using the private key (e.g., through system login validation). In such embodiments, the genetic identification information management service provider does not process, retain, or have access to any unencrypted data. In other or further embodiments, the member may share the public keys with one or more trusted persons (e.g., family, one or more friends) or organizations through the genetic identification information management system. The public key provides the trusted person(s) or organization(s) with the ability to decrypt the member's genetic identification data and/or other PII in case of need.

Several levels of decryption may be available in the genetic identification information management system to enable separate access to common or basic PII, secure documents, and/or the genetic identification information. For example, all such information may be encrypted using a conventional encryption algorithm, thus providing a first level of decryption. Certain information (e.g., secure documents and/or the genetic identification information) may be compressed (e.g., prior to encryption), thus providing a second level of decryption to enable separate access to such information. Furthermore, the member's genetic identification information may first be converted to a digital (e.g., p-character) format, as described herein, before compression and/or encryption, thus providing a third level of decryption to enable separate access to the member's genetic identification information.

Using the decryption algorithms and segments of the encrypted DNA sequence (i.e., the “seed), real-time validation challenge tokens may be generated. The challenge tokens may comprise a code such as a QR code, a bar code, or an authorization code. These tokens may be exchanged programmatically, scanned, typed, or communicated verbally to validate the user's/member's identity based on the user's/member's DNA sequence. This validation may be a part of a multi-factor identification solution, as described herein.

In this manner, the encrypted DNA information cannot be matched to familial DNA, which may be accessible to others or in the public domain. Accordingly, it is not possible to obtain the user's/member's DNA information or genetic identity from information that is available to others.

The PII management system may allow members or users who do not provide a DNA sample to replace the DNA sequence encryption/decryption seed with a numeric string representation from a facial ID scan, fingerprint scan, or other digital representation of a unique personal identifier.

In some cases, the genetic identity will be requested by a third-party authority such as governmental agency, a corporate entity, or a social group. The genetic identification information management system or architecture may segregate data repositories (e.g., blockchain registers) so that the individual may retain SSI over data that the individual designates as SSI (e.g., stored in one repository or an SSI segment of a repository), and the third-party authority may access certain information to be shared (e.g., that is stored in a separate repository or in a “sharable” segment of the one repository).

The authorized device data and genetic identification code (e.g., QR code or bar code) may also be modified by the authority on a real-time basis in order to provide a validation key that certifies the currency (e.g., “most recent” status of the data) and/or validity of the genetic identification code as of the time of use. Implementations include placing or embedding the genetic identification code or other information to be presented using an electronic communication device, or on physical items such as ID badges, wallet cards, RFID tags, or wearables. Such physical items may also include an authority validation code placed thereon or embedded therein.

The genetic identification information management system or architecture may include an application programming interface (API) that enables genetic identification and other information in the management system or architecture and available to the authority to be exchanged, in a two-way manner, with other established information systems of, or controlled by, the authority. The API respects (e.g., cannot bypass) the encryption/decryption algorithms, and allows access to and exchange of only that information allowed by the individual member/registrant to be shared with the authority. In some embodiments of the two-way API, the authority may have the ability to push an encrypted security key to the genetic identification information management system or architecture. This allows the authority to include a software key for authority-controlled data, such as the individual's genetic identification code (e.g., QR code) or the underlying genetic identification data, stored documents, or other data that the individual cannot access. This would be utilized in the event of suspected compromise of the login validation, such as lost/stolen device/ID, suspected duress, or separation from the authority.

To encourage participation in a public permission genetic identification information blockchain network, a reward and data sharing process may be used. FIG. 6 shows a flow chart 500 for an exemplary reward and data sharing process, which begins by inputting personal data at 502, and setting sharing controls on the personal data at 504. These actions can also take place during user registration 110 (FIG. 1).

The reward and data sharing process 500 may share some or all personal data with authorized entities at 506, such as governmental authorities, health care service providers, security service providers, insurance companies, etc. The specific parties or types of parties with whom the information may be shared is also defined at 506. The authorizations and/or permissions for sharing the personal information with other parties and/or entities are written into the blockchain ledger at 520. When the user/registrant provides at least some personal information, the process 500 provides a reward to the user/registrant at 508 via a digital wallet 518. The reward may be commensurate with the amount and/or type of information shared and/or the number and/or types of third parties given access to the information.

At various times, the process 500 may initiate a user participation event such as a survey questionnaire or a shopping or travel opportunity at 514. When the user/registrant participates in the user participation event, the process 500 provides another reward at 516. The reward may be commensurate with the amount of money spent and/or the total number of user participation events in which the user has participated.

The rewards may be deposited in the digital wallet 518 as cryptotokens, other electronic currency, or as discounts on products or services offered by participating sellers or providers. The transactions at 506, 508, 514 and 516 are recorded on the blockchain ledger 520.

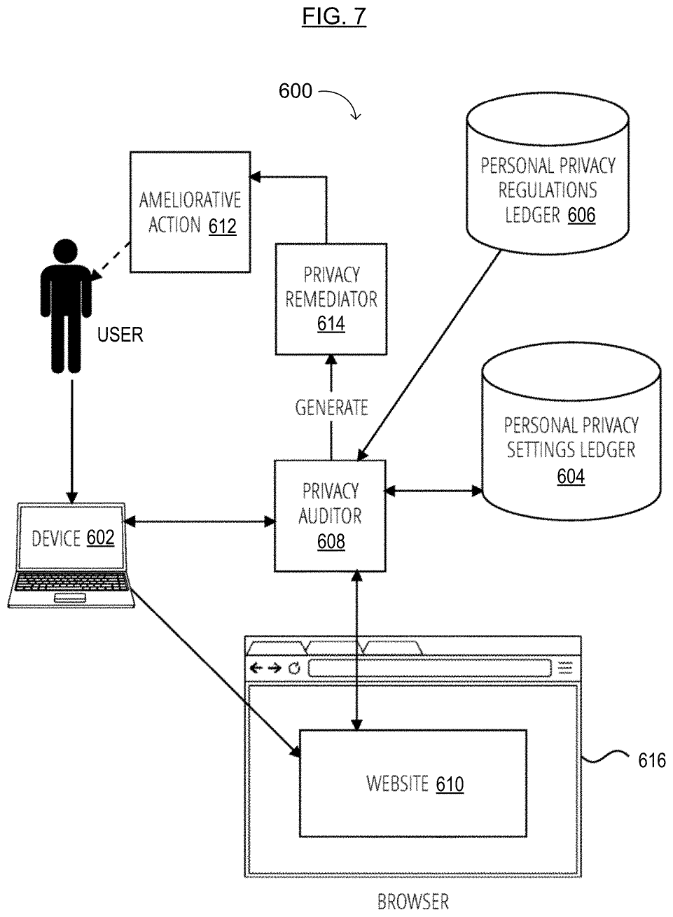

Referring now to FIG. 7, a genetic information privacy system 600 may comprise an electronic communication device 602, a personal/genetic information privacy settings ledger 604, a personal/genetic information privacy regulations ledger 606, a privacy auditor 608, a website 610, an ameliorative action 612, a privacy remediator 614, and a browser 616. The web portal 230 (FIG. 3) may comprise the electronic communication device 602 and the browser 616.

A universal privacy settings/opt-in/opt-out client (a “universal client”) allows a user to connect to an application program interface (API) for one or more different sites that have the user's data. The universal client orchestrates curation of privacy settings and overall opting in or out of any sites that the user selects or that are provided by default. It allows the user to select total or partial opt-ins or opt-outs where the user has granular control when they may wish to allow some uses of data and access to data, but restrict others. When a user is calibrating their privacy and data settings, a company or site or distributed application may provide reasons and incentives for the user to allow access to certain data (see the discussion of FIG. 6 above). This allows users to have simultaneous global control over their personal data, while enabling the user to receive compensation and/or services for the use of their personal data, thus allowing companies to have access to better data.

The user may retain global control over their personal data and enable others to access and/or use certain of their personal data by maintaining a universal profile with a personal privacy policy, which may be applied to company privacy policies. In some cases, the system may automatically resolve conflicts between the personal privacy policy and the company privacy policy or policies. Common settings across sites may have a unified view, and unique settings per site may be labeled with a site identifier. This allows the user's data and privacy settings to remain consistent across sites where common data and settings are used and uniquely where required by individual sites.

The user may authenticate the privacy system 600 into web sites and decentralized services and authorize its access to the sites utilizing the user's credentials. Where blockchain IDs are used, the privacy system may similarly operate on behavior of the user. For example, after the user has installed the system's user portion(s) (e.g., using electronic communication device 602), a user may use a mobile device or computer (e.g., similar or identical to device 602) to go to a site 610 or decentralized service, such as Facebook, Steemit, or STR-ID (Lewes, Del.). The first time the user does this, the system 600 may automatically generate a pop-up window or notification and ask the user for their settings, allowing the system to auto-configure based on the user's online behavior. This allows the user greater freedom to use the most efficient software for their purpose, since the user is not forced to access sites 610 through the system 600, which runs parallel to, or in the user's browser 616. The system 600 may run in the background (like a daemon) and monitor sites 610 unobtrusively.

The system 600 may then see that the user accessed a site 610 or decentralized service that had in the past contained the user's personal data. The system 600 may inquire how the user would like to have their data managed on that site 610. The system 600 may allow the user to also configure when the system 600 is running. The system 600 may, for example, allow the user to toggle the system off and on, or allow the user to set specific instances or sites which should be explicitly included or excluded (i.e. “whitelisted” or “blacklisted”), or allow the user to “suspend” protection if desired.

When that site 610 or decentralized service is accessed in the future, the system 600 enforces the privacy settings in the ledger 604 through the browser 616 or interface used to access that site 610. Otherwise, the site 610 may automatically (re)configure the user's profile. For example, a user's Facebook profile may be automatically configured to reflect the user's preferences for Facebook's website (or decentralized service).

The system 600 may synchronize with privacy settings in the personal/genetic information privacy settings ledger 604 that the user has changed manually to resolve and/or approve conflicts. When the system 600 connects to the privacy settings or opt-in/opt-out settings of the site 610 or the decentralized service, the system 600 may evaluate such settings to see if any changes were made. The system 600 may access a site 610 or decentralized service through an API or more directly through “web scraping” and may employ the user's ID and other personal information (e.g., in the ledger 604) to gain access. The system 600 may utilize an intermediary to analyze the settings and do a manual translation until the system 600 can gain access to the site 610. The system 600 may be configured with a country's privacy laws (e.g., recorded in the personal/genetic information privacy regulations ledger 606) and may monitor information on websites and decentralized services for compliance with both the user's settings as well as the privacy laws of that jurisdiction.

In the system 600, the privacy auditor 608 may scan websites 610 for the user's personal information. For example, the privacy auditor 608 may configure the browser 616 with a concept filter (not shown), and the browser 616 may then analyze data on the site 610. The browser 616 may then detect information on the site 610 that is not congruent with the personal privacy settings ledger 616 and/or the personal privacy regulations ledger 606. The browser 616 may then notify the privacy auditor 608, and the privacy auditor 608 may then notify the privacy remediator 614. An alert generator (e.g., in the device 602 and/or browser 616) receives the notification and generates an ameliorative action 612. The ameliorative action 612 may comprise, for example, accessing the incongruent data on the site 610 (e.g., via an API) and correcting the incongruence, or populating and transmitting a form cease-and-desist letter to the host and/or owner of the site 610 or decentralized service. The browser 616 may monitor and “crawl” websites for the registrant's personal information, and may access and monitor personal information on distributed applications, such as blockchain-based distributed services, sites, and applications.

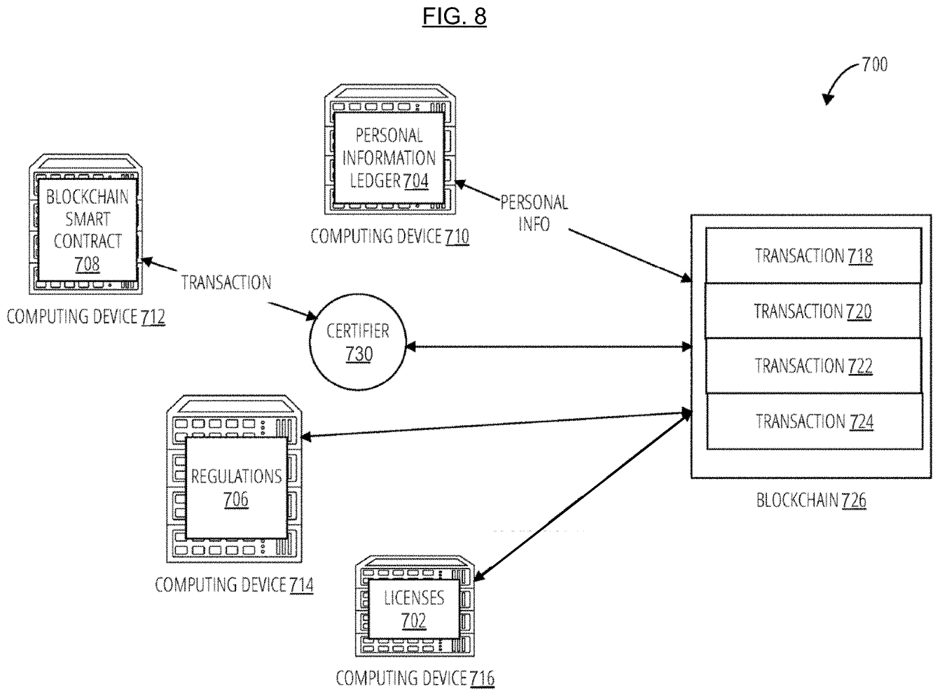

FIG. 8 shows an exemplary distributed blockchain storage network 700 comprising a computing device 710 storing a personal/genetic information ledger 704 (e.g., similar or identical to ledger 520, FIG. 6), a computing device 712 storing a blockchain smart contract 708, a computing device 714 storing regulations 706 (e.g., in the form of a personal/genetic information privacy regulations ledger), a computing device 716 storing one or more licenses 702, a certifier 730, and a plurality of transactions 718, 720, 722 and 724 in a blockchain 726. The computing device 710 transmits personal information ledger 704 (as needed) to the blockchain 726 and vice versa. The computing device 712 transmits the blockchain smart contract 708 (as needed) to the blockchain 726 via the certifier 730 and vice versa.

The computing device 714 records the regulations 706 on the blockchain 726, and the computing device 716 records the licenses 702 on the blockchain 726. The licenses 702 may include certain personal information, such as driver's license information, passport information, professional licenses, etc., and/or any licenses or third-party permissions necessary to share the registrant's genetic identification information and/or other personal information with others. The licenses 702, regulations 706, blockchain smart contract 708 and personal information ledger 704 may be recorded on the blockchain 726 as the transaction 718, the transaction 720, the transaction 722, and the transaction 724, respectively. The blockchain 726 may be distributed on or among the computing devices 710, 712, 714 and 716.

An Exemplary System

Similar to the present method, the present system may be divided into two parts or sections: a genetic testing part or section, and a digitalization/identification information management part or section. Referring back to FIG. 3, the genetic testing part or section of an exemplary system 200 comprises one or more DNA (e.g., STR) testing kits 210, and the digitalization/identification information management part or section of the exemplary system 200 comprises a DNA or genetic information digitization system 220, a web portal 230, and a mobile application 240, as described herein.

In one example, the DNA test kit 210 may comprise a DNA home sample collection kit. DNA home sample collection kits may be commercially available from companies that produce STR analysis kits, such as Thermo Fisher, Promega, and Qiagen, but can also be readily assembled. A typical kit 210 includes a tube or sample cup with a cap or lid for collecting the DNA sample, and detailed written instructions for the user to properly collect the sample and return it to the test facility (e.g., analysis lab). Detailed procedures for forensic DNA sample collection are well-known and widely available (see, e.g., Tan, E., “Sample Collection System for DNA Analysis of Forensic Evidence: Towards Practical, Fully-Integrated STR Analysis,” NIJ Award 2008-DN-BX-K010, Document No. 236826, December 2011, National Criminal Justice Reference Service, Rockville, Md.; http://www.geneticprofiles.com/procedure/; and https://blog.puritanmedproducts.com/how-to-collect-dna-evidence, among others). Optionally, the kit 210 includes a swab (e.g., for procuring a saliva sample from inside the registrant's mouth or a mucus sample from inside the registrant's nose, etc.) or an absorbent paper or cotton pad (e.g., for absorbing a blood sample following a pin or needle prick of the registrant's fingertip, heel, etc.). The kit 210 typically also contains an envelope or box for shipping the sample to lab or test facility for analysis, and a container (e.g., a box or envelope) in which all of the kit components are placed.

Alternatively or additionally, the test kit 210 may comprise a commercially-available STR analysis kit for use in the test facility or analysis lab, such as those available from Thermo Fisher, Promega Corporation, Qiagen and others, described herein with respect to STR analysis 150 in FIG. 1. However, the STR analysis kit is not for home use, and typically does not include components for sample collection.

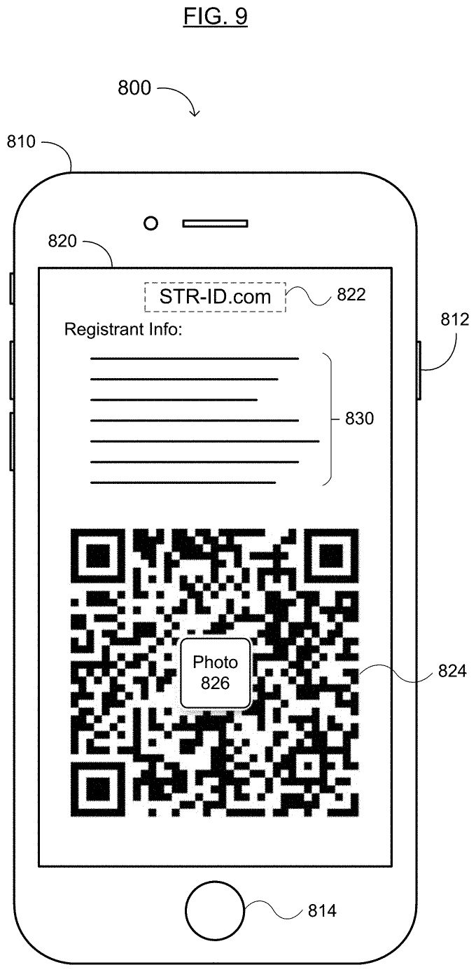

The mobile application 240 may be installed on an electronic communication device 800 (FIG. 9) such as a smartphone 810. The smartphone 810 in FIG. 9 is displaying on its screen 820 personal information 830 and genetic identification 824 accessed through a secure application or website 822. The personal information 830 is that of the registrant, and may include the registrant's name, residence address, birthdate, social security number or other government-issued identifier, driver's license number and other information associated with the registrant's driver's license, and/or passport number and other information associated with the registrant's passport, among other personal information as described herein. The genetic identification 824 may be in the form of a QR code (as shown), but may take other electronically readable or scannable forms as described herein. In some embodiments, to further facilitate identification of the registrant, the genetic identification 824 (or, if desired, the personal information 830) may further include a photograph 826 of the registrant. The smartphone 810 may further include features such as an on-off button or switch 812 and an application-closing/switching and/or screen-changing button 814, among others.

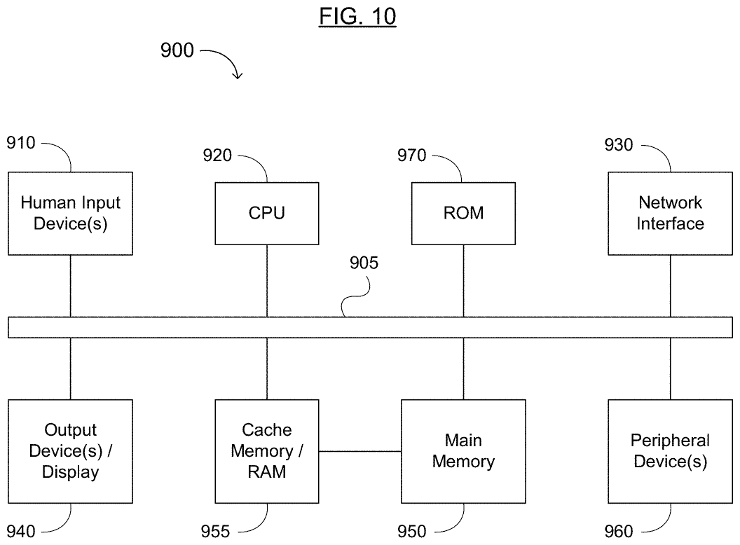

The web portal 230 may be included on a webpage (e.g., 610, FIG. 7) accessible through a browser (e.g., 616). For example, registration may be conducted using the web portal 230, which can be accessed by the smartphone 810 or on an alternative electronic communication device 900 as shown in FIG. 10. The electronic communication device 900 may be in the form of a personal computer, workstation, tablet computer, personal digital assistant, or the like.

FIG. 10 shows a basic architecture for the electronic communication device 900, including components such as one or more human input devices 910, a central processing unit (CPU) 920, a network interface 930, an output and/or display device 940, main memory 950, cache memory and/or random access memory (RAM) 955, one or more peripheral devices 960, and a read-only memory (ROM) 970. These components communicate with each other over one or more busses 905. The architecture of the electronic communication device 900 is largely conventional.

For example, the human input device(s) 910 may comprise a keyboard (e.g., a stand-alone or virtual keyboard), a mouse, a microphone (working together with speech recognition software stored in the main memory 950 and executed by the CPU 920), finger print reader, facial recognition system, etc. The network interface 930 may enable communications between the electronic communication device 900 and a home network, an intranet, a data and/or voice network, and/or the Internet, and may be wired or wireless. The output and/or display device 940 may comprise a monitor, display screen, television, one or more speakers, etc. The main memory 950 may comprise a magnetic or nonvolatile (e.g., flash) hard drive, configured to store software programs, data, user preferences, etc. The cache memory and/or random access memory (RAM) 955 may temporarily store recently used programs, routines or subroutines of programs, data, etc. for more facile use of such data, programs and (sub)routines. The peripheral device(s) 960 may comprise devices such as a printer, an external memory, speakers, a wireless receiver (e.g., from other devices such as a keyboard, mouse, etc.), a camera, a smartphone or tablet computer, etc. The read-only memory (ROM) 970 may store information and programs that generally cannot be erased or reprogrammed, such as device booting or start-up information, disk operating system (DOS) software, device configuration settings, etc.

The invention may be implementable in any of a variety of different types of blockchain networks. In particular, the present system may be implemented using a public blockchain network, a private blockchain network, a permissioned blockchain network, a consortium blockchain, or a combination thereof. Examples of such blockchain networks and the functions and transactions that they carry out are shown in FIGS. 5-8 and discussed in some detail above.

A private blockchain network, similar to a public blockchain network, is a decentralized peer-to-peer network, with the difference that one organization governs the network. (In a public blockchain network, no one organization or entity governs the network.) The organization that governs the private blockchain network controls who receives permission to participate in the network, executes a consensus protocol, and maintains the shared ledger. Alternatively, the organization that governs the private blockchain network may also control who executes a consensus protocol and maintains the shared ledger. Depending on the use case, this can significantly boost trust and confidence between participants. A private blockchain can be run behind a firewall and be hosted on-premises.

Businesses that set up a private blockchain often set up a permissioned blockchain network. Public blockchain networks may also be permissioned blockchain networks. This may place restrictions on (1) who may participate in the network and (2) the transactions in which certain participants may participate. Participants need to obtain an invitation or permission to join a permissioned blockchain network.

Multiple organizations can share the responsibilities of maintaining a blockchain. These organizations (which may be pre-selected) determine who may submit transactions or access the data stored in a ledger. A consortium blockchain network is ideal when all participants need to be permissioned and have a shared responsibility for the blockchain.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A method of obtaining and controlling genetic identification information, comprising:
a) providing personal information of a registrant to a secure website using a first electronic communication device;
b) taking a genetic material-containing sample from the registrant;
c) providing the genetic material-containing sample to a genetic material analysis facility;
d) analyzing short tandem repeat (STR) regions of the genetic material at a plurality of loci to produce a genetic identity for the registrant;
e) recording the personal information and the genetic identity in a blockchain ledger; and
f) enabling the registrant to display on a second electronic communication device a code corresponding to the genetic identity, wherein the first and second electronic communication devices are a same device or different devices.

2. The method of claim 1, wherein said personal information comprises at least two of a name, an address, a government-issued identification number, and a photograph of the registrant.

3. The method of claim 1, further comprising encrypting the personal information and the genetic identity of the registrant prior to recording the personal information and the genetic identity in the blockchain ledger.

4. The method of claim 1, further comprising registering said registrant for a service comprising the STR region analysis, the personal information/genetic identity recordation, and genetic identity code display enablement.

5. The method of claim 1, wherein the first and second electronic communication devices are independently selected from a smart phone, a personal computer, a tablet computer, and a work station.

6. The method of claim 1, wherein taking the genetic material-containing sample from the registrant comprises collecting the registrant's saliva in the vial or tube, swabbing an inner surface of the registrant's mouth or nose, or pricking/puncturing the registrant's skin and collecting one or more drops of the registrant's blood on a swab or piece of absorbent paper, and the method further comprises (i) the registrant certifying or confirming that the registrant collected the DNA sample or (ii) a third party certifying or confirming that the third party has authority to collect the DNA sample of the registrant.

7. The method of claim 1, wherein providing the genetic material-containing sample to the genetic material analysis facility comprises shipping the genetic material-containing sample to the genetic material analysis facility in an envelope, sleeve, tube or box.

8. The method of claim 1, wherein analyzing the STR regions of the genetic material comprises extracting DNA from the genetic material, amplifying the DNA at multiple STR loci, separating and sizing amplified STR alleles, and interpreting a profile of the separated and sized STR alleles.

9. The method of claim 1, further comprising allowing the registrant to access entries in the blockchain ledger containing the personal information and the genetic identity.

10. The method of claim 1, further comprising enabling the registrant to authorize third parties to access the code on a third electronic communication device, wherein third electronic communication device is identical to, same as, or different from one or both of the first and second electronic communication devices.

11. The method of claim 1, further comprising accessing the code using one of the first and second electronic communication devices.

12. The method of claim 1, further comprising authenticating an identity or personal information of the registrant using the registrant's genetic identity.

13. A system for obtaining and controlling genetic identification information, comprising:
a) a genetic material sampling kit, comprising:
i) a sealable container configured to sealably contain a sample containing genetic material of a registrant,
ii) written instructions for taking the sample from the registrant and placing the sample in the sealable container, and
iii) a pre-addressed envelope or box for sending the sample in the sealable container to a genetic material analysis facility;
b) a short tandem repeat (STR) analysis kit, comprising:
i) a plurality of primers for copying STR regions in the genetic material at a plurality of loci, and
ii) a mixture containing a genetic material polymerase, buffers, and dNTPs necessary to amplify the STR regions, compare the amplified STR regions with similar genetic identification information and produce a genetic identity for the registrant;
c) a first electronic communication device configured to enter personal information of the registrant to a secure website; and
d) a second electronic communication device configured to record the personal information and the genetic identity in a blockchain ledger; and
e) a third electronic communication device configured to display a code corresponding to the genetic identity,
wherein the second electronic communication device is different from the first and third electronic communication devices, and the first and third electronic communication devices are a same electronic communication device or different electronic communication devices.

14. The system of claim 13, wherein said sealable container comprises a sealable plastic bag or a vial or tube with a cap or lid configured to seal an opening in the vial or tube.

15. The system of claim 13, wherein said STR analysis kit further comprises a gel electrophoresis cassette or tray and a gel, or a capillary electrophoresis capillary, configured to separate the amplified STR regions by size.

16. The system of claim 13, wherein said primers include a fluorescent or luminescent label.

17. The system of claim 13, further comprising a genetic analyzer.

18. The system of claim 13, wherein the first electronic communication device comprises a personal computer or a smart phone.

19. The system of claim 13, wherein said second electronic communication device comprises a personal computer, a workstation, or a server.

20. The system of claim 13, wherein said third electronic communication device is further configured to access the code from the blockchain ledger.
